(19) United States
(12) Patent Application Publication
(10) Pub. No.: US 2026/0070719 A1
(43) Pub. Date: Mar. 12, 2026

(54) A PACKAGE COMPRISING A BAG AND AT LEAST TWO ABSORBENT ARTICLES

(71) Applicant: Essity Hygiene and Health Aktiebolag, Göteborg (SE)

(72) Inventors: Hanna THERSTOL, Göteborg (SE); Matteo VANUZZO, Lucca LU (IT)

(73) Assignee: Essity Hygiene and Health Aktiebolag, Göteborg (SE)

(21) Appl. No.: 19/106,248

(22) PCT Filed: Nov. 16, 2022
(86) PCT No.: PCT/EP2022/082081

§ 371 Date: Feb. 25, 2025

(51) Int. Cl. B65D 65/46; A61F 13/551; B32B 1/00; B32B 29/00; B65D 33/16; B65D 33/18; B65D 85/62

(52) U.S. Cl. CPC B65D 65/466; A61F 13/5511; A61F 13/55115; A61F 13/5514; A61F 13/55145; B32B 1/00; B32B 29/005; B65D 33/1691; B65D 33/18; B65D 85/62; B32B 2250/02; B32B 2250/26; B32B 2307/718; B32B 2439/06; B65D 2565/385

(57) ABSTRACT

A package including a bag and at least two absorbent articles arranged in the bag, the bag being made of a first paper material, wherein the bag has a sealed bottom, a top seal, and a peripheral wall extending between the bottom and the top seal. The peripheral wall includes a front wall, an opposite rear wall, first and second side walls connecting the front and rear walls, a first top surface extending between the top seal and the front wall, and a second top surface extending between the top seal and the rear wall. The top seal has been formed by adhering inner surface top portions of the peripheral wall to one another. The package further includes at least one adhesive patch configured to hold the top seal folded onto one of the first and the second top surfaces.

### TECHNICAL FIELD

The disclosure relates to a package comprising a bag and at least two absorbent articles arranged in the bag, such as disposable personal hygiene absorbent articles in the form of incontinent shields, sanitary napkins, panty liners, diapers, or the like.

### BACKGROUND

Disposable personal hygiene absorbent articles, for example diapers or absorbent pads such as incontinent shields or sanitary napkins, are typically provided in multipacks comprising one or more stacks of products arranged in a plastic bag. Although plastic materials are versatile and offer a range of advantages such as low weight, high flexibility and easy sealing, plastic packaging materials may also be associated with disadvantages such as low degradability and recyclability, production of toxic gases during combustion, littering, etc. Consequently, there is currently a strive to replace plastic packaging by more sustainable and/or renewable alternatives, such as paper materials.

### SUMMARY

An object of the disclosure is to provide an, in at least some aspect, improved package comprising a bag and at least two absorbent articles. The object is achieved by a package according to claim 1.

Hence, a package comprising a bag and at least two absorbent articles arranged in the bag is provided, wherein the bag is made of a paper material. The absorbent article may be a disposable personal hygiene absorbent article such as an incontinent shield, a sanitary napkin, a panty liner, a tampon, a diaper, or the like.

Disposable sanitary articles, such as sanitary napkins, panty liners or the like are typically intended to absorb body liquids, such as urine and blood.

Disposable diapers can be pants diapers, open diapers, also called all-in-one diapers or belted absorbent products, which allows a wearer or caregiver to apply the diaper in a standing position. The diapers can be diapers for babies, young children or adults.

A belted absorbent product, i.e. a belt product, typically has a belt attached integrally with a chassis of the absorbent product. The belt may have two belt portions extending on either side of the rear end of the chassis. The two belt portions are intended to be fastened around the waist of the wearer, whilst the front end of the absorbent section hangs down between the legs of the wearer. Once the belt portions have been joined together, by a fastener or such as a fastening tab, the wearer can reach between his or her legs to draw up the absorbent section between the legs and to attach the free end of the absorbent section to the belt portion, by second fasteners such as fastener tabs. Similarly, the product can be made so that the belt is fastened to the front portion of the product and is secured around the back of the wearer. In this case, the absorption section will be hanging down in the front and will be secured to the belt in the back. This type of product is particularly useful for caregivers who care for patients that may have dementia or the like.

The term “disposable” is used to describe absorbent products which generally are not intended to be laundered or otherwise restored, or reused as an absorbent product, e.g., they are intended to be discarded after a single use and may also be configured to be recycled, composted or otherwise disposed of in an environmentally compatible manner.

The absorbent article may alternatively be a reusable and washable absorbent article such as a washable and reusable sanitary article, such as a sanitary napkin, panty liner or the like, or a washable and reusable absorbent undergarment for urine, vaginal fluids and/or menstrual fluids, or a diaper for urine and feces. The washable and reusable diaper may be fully washable or be a diaper having a washable outer shell adapted for receiving an absorbent assembly, either a reusable absorbent assembly or a disposable absorbent assembly.

Preferably, the absorbent article may be an absorbent pad such as an incontinent shield, a sanitary napkin, or a panty liner.

The bag has a sealed bottom, a top seal, and a peripheral wall extending between the bottom and the top seal. The peripheral wall comprises a front wall, an opposite rear wall, and first and second side walls connecting the front and rear walls. The peripheral wall further comprises a first top surface extending between the top seal and the front wall, and a second top surface extending between the top seal and the rear wall. The top seal has been formed by adhering inner surface top portions of the peripheral wall to one another. The package further comprises at least one adhesive patch configured to hold the top seal folded onto one of the first and the second top surfaces. The at least one adhesive patch may, e.g., be a sticker or an adhesive tape. The at least one adhesive patch may have any suitable shape, such as rectangular, square, oval, oblong, triangular, circular, etc.

The package according to the disclosure has a gable-like top achieved by adhering inner surface portions of the peripheral wall to one another after filling the bag with the absorbent articles. By providing an adhesive patch that holds the top seal folded onto one of the first and the second top surfaces, the package can assume the basic shape of a rectangular block, i.e., with a flatter top than it would otherwise have. This is advantageous from a packaging point of view, since the packages with the flatter top are on the one hand more stable when stacked onto one another, and on the other hand occupy less space. Furthermore, the package is less prone to being damaged when the top is flattened by means of the at least one adhesive patch. Damaged packages may otherwise be a problem that arises when moving from a plastic packaging material to a paper material, since paper materials are generally more fragile and less flexible than plastic packaging materials.

Furthermore, the package disclosed herein achieves the above advantages without having to use a hot-melt adhesive for attaching the top seal to the top surface, such as by applying hot-melt adhesive dots to one of the surfaces and pressing the other surface onto it. In this way, production technical problems in connection with application of the hot-melt adhesive as well as problems associated with recycling of paper material that comprises hot-melt adhesive are reduced.

The adhesive patch is further beneficial for providing an additional sealing at a weak portion of the top seal. Such a weak portion with seal integrity problems may arise along a mid-portion of the top seal, in an area between opposite “wings”, or fins, of the gable-like top. This can be due to the uneven thickness of the top seal when the inner surface portions of the peripheral wall are sealed to one another after filling the bag with the absorbent articles. The top seal can be accomplished by pressing two seal bars against each other with the inner surface portions of the peripheral wall to be adhered together between the seal bars. The adhesive, for example a hot melt adhesive which is applied to the bag, melts and attach the surface portions to each other and form the top seal. Due to an uneven thickness, which is dependent on the outer dimensions of the bag, a weak portion can be created. The adhesive patch may be positioned on the top seal such that it covers the weak portion, i.e., half-way between the first and second side surfaces of the bag.

Optionally, the at least one adhesive patch comprises a non-adhesive front surface and a back surface, wherein an adhesive layer is provided on at least a portion of the back surface. The adhesive layer may cover the back surface in its entirety, or it may cover only a portion of the back surface such that a non-adhesive part is provided, e.g., for gripping the adhesive patch to remove it. The adhesive may also be discontinuously distributed on the back surface, for example, as lines in the form of stripes or in a spiral pattern. The stripes or spirals may be at a distance from each other or overlapping. The adhesive is not limited to being distributed as lines. As an alternative, it may be distributed as dots, or islands, with adhesive regularly or unregularly distributed over the back surface as long as sufficient adherence is accomplished. The peel strength should be high enough to maintain the package in a folded configuration, but not so high that it exceeds the tensile strength of the bag.

The front surface may comprise a decorated surface, such as a printed surface.

The adhesive patch may also have an embossment. The embossment can be made to give the adhesive patch a nice appearance. The embossment may also be an aid for visually impaired and blind persons who are going to use the products inside the package. For example, a portion of the adhesive patch may have embossed alphabet symbols representing the letters of the conventional Roman alphabet or embossed numeral symbols representing the conventional Arabic numerals or other symbols, showing for example the absorption capacity with embossed drops, or symbols indicating where to open the package.

Optionally, a first portion of the at least one adhesive patch is adhered to the top seal and a second portion of the at least one adhesive patch is adhered to the one of the first and second top surfaces onto which the top seal is folded, and/or to one of the front and rear walls. Hence, at least the second portion of the at least one adhesive patch is used for keeping the top seal folded onto the top surface. Preferably, the at least one adhesive patch has an elongated shape with a long side extending perpendicularly to the top seal. Preferably, when a single adhesive patch is provided, it may be positioned half-way between the first and second side surfaces of the bag so as to cover a potentially weak area of the top seal. A length of the at least one adhesive patch may be selected so that the at least one adhesive patch adheres both to one of the top surfaces and to the adjacent one of the rear wall and the front wall, or such that the at least one adhesive patch adheres only to the top surface onto which the top seal is folded. Of course, the first portion of the at least one adhesive patch may also extend onto the other one of the first and second top surfaces. Optionally, the at least one adhesive patch may extend all the way from the rear surface, over the second top surface, the top seal and the first top surface, and onto the front surface. Depending on the length of the top seal, the at least one adhesive patch may extend from the top seal and onto the front or back surface, or extend all the way from the rear surface, over the second top surface, the top seal and onto the front surface. That is, the folded top seal may overlap the first or the second top surface partly of fully. It might even be cases when the folded top seal overlaps partly with the front or the rear surface. If the at least one adhesive patch has an embossment, it may be provided on the second portion. The embossment may then reduce the adherence of the second portion to the package and make it easier to open.

Optionally, a first portion of the at least one adhesive patch is adhered to the top seal, and a second portion of the at least one adhesive patch is adhered to one of the first and second side walls. In this case, the at least one adhesive patch preferably has an elongated shape with a long side extending in parallel with the top seal along the first portion of the at least one adhesive patch.

Optionally, a third portion of the at least one adhesive patch is adhered to the other one of the first and second side walls. In this case, the adhesive patch has an elongated shape with the first portion extending along the top seal, the second portion being attached to one of the first and second side walls, and the third portion being attached to the other one of the first and second side walls.

In the above embodiments, the same adhesive may be used for the adhesive layer in the first, second and, if applicable, third portions of the at least one adhesive patch. The area of the first portion attached to the top seal is in that case preferably larger than the area of the second and/or third portion(s), so that the at least one adhesive patch is less at risk of releasing from the top seal than from the other surface of the bag in connection with opening. Alternatively, a different adhesive may be used for the first portion than for the second and/or third portion/s. The first portion may comprise an adhesive that securely attaches the at least one adhesive patch to the top seal of the bag. The second and/or third portion(s) may then comprise an adhesive that forms a releasable bond with another surface of the bag.

Optionally, the at least one adhesive patch comprises a non-adhesive gripping portion configured to be gripped by a user for releasing at least the second portion of the at least one adhesive patch from the bag. The gripping portion is preferably located adjacent to the second portion of the at least one adhesive patch and/or adjacent to the third portion of the at least one adhesive patch. The at least one adhesive patch may thereby remain attached to the bag while allowing the bag to be opened.

Optionally, the at least one adhesive patch comprises a first adhesive patch and a second adhesive patch. Hence, at least two adhesive patches are provided. The second adhesive patch may have the same properties as the first adhesive patch. The second adhesive patch may in some cases have a different shape than the first adhesive patch. By using two adhesive patches, the flatness of the top may be improved.

Optionally, the first adhesive patch is attached to the top seal close to the first side wall, and the second adhesive patch is attached to the top seal close to the second side wall. By “close to the first side wall” is herein intended that the first adhesive patch is positioned between the first side wall and a mid-portion of the top seal, located halfway between the side walls. By “close to the second side wall” is intended that the second adhesive patch is positioned between the second side wall and the mid-portion of the top seal. The first and second adhesive patches are hence preferably interspaced. This configuration is advantageous for packages having a relatively long top seal.

Optionally, the adhesive layer is a self-adhesive layer, i.e., a layer sticks without wetting. The adhesive layer may comprise any suitable adhesive that provides desired peel strength, quick-stick, releasability from the package, and coat weight properties. The adhesive can, for example, be a hotmelt adhesive which is coated onto a backing material of the adhesive patch by any of the well-known hotmelt coating processes.

A peel strength of the adhesive patch should be high enough to maintain the package in a folded configuration, but not so high that it exceeds the tensile strength of the bag, or of a landing member provided thereon, to ensure good resealability.

The adhesive may be a biobased adhesive. It may also be a biodegradable adhesive. It may be made from a renewable resource and/or may be compostable.

The package may be provided with a landing member (or “landing surface”), if for example the first paper material of the bag is very thin. The landing member may have an adherence surface which is the surface onto which the adhesive of the adhesive patch is resealably adhered.

The properties of an overall fastening system, including the properties of the adhesive on the adhesive patch and the properties of the landing member or the paper material, are important design criteria in the performance of the package disclosed herein. The fastening system needs to provide a bond that can be opened to remove the absorbent articles for use by the wearer without destruction or damage to the landing surface or the paper bag, and preferably a bond that can also be refastened.

Optionally, the self-adhesive layer is configured to allow resealing of the bag by the at least one adhesive patch. Hence, the self-adhesive layer comprises a resealable adhesive. In this way, a user may easily reseal the package such that it acquires a compact shape after removing an absorbent article. Since paper material packages are generally more volumetric and stiffer than plastic packages, they tend to bounce back when folded down after opening. By using a self-adherent layer that allows resealing, compacting of the package after opening to occupy less space is facilitated and a better protection of the absorbent articles therein can be achieved. This is particularly important in packages comprising two stacks of absorbent products, since an excessive amount of material may remain after having used up the upper stack.

By a “resealable adhesive” as used herein is meant an adhesive that provides that an object may be attached to a surface using such an adhesive and subsequently detached from that surface causing minor or no disruptions of the object and the surface. Furthermore, an object that has been peeled off may be reattached to a surface again.

The resealable adhesive may be a pressure-sensitive hotmelt adhesive. Such an adhesive provides unlimited open time, meaning that the resealable adhesive can bond to another substrate at any time. Such a resealable adhesive may provide “post-it-like” adhesive properties. Thus, the package may be re-closable. Accordingly, the pressure-sensitive hotmelt adhesive used with the package is one which has a very high self-adhesion, but which can be readily separated or released from other materials.

Optionally, the top seal together with the first and/or second top surfaces of the package form a planar or an essentially planar surface extending in parallel with the sealed bottom when at least the at least one adhesive patch holds the top seal folded onto one of the first and the second top surfaces. A brick-formed package which is easy to pack and transport in multi-packs comprising several packages within a larger box is thereby provided.

Optionally, the at least one adhesive patch comprises a backing material in the form of a second paper material.

Optionally, the backing material of the at least one adhesive patch, i.e. the material on which the adhesive layer is provided, may be any type of tape backing materials well known in the art. For example, polyester films, polypropylene films, paper backings, or other materials which provide the required strength to be useful as part of an adhesive patch used to maintain the package in a folded configuration before opening the package and to maintain the package in a folded configuration after opening it for storage, are suitable for use as the backing material. The backing material may be a biobased material. It may also be a biodegradable material. It may be made from a renewable resource and/or may be compostable.

Optionally, the first paper material of the bag has a basis weight between 50 and 90 g/m² as measured according to the ISO 536 standard. A paper material having a basis weight within this range is sufficiently thick and strong to provide accurate protection for the absorbent pads within the bag against environmental pollution and can also withstand stresses arising during shaping of the bag and loading of absorbent pads into the bag. The paper material may be a single layer (one ply) or several layers.

By a paper material, i.e. when referring to the first paper material and the second paper material, is herein to be understood a material produced by pressing together moist fibres, dewatering and drying the fibres into preferably flexible sheets. The paper material may be an unbleached paper material or a bleached material. The fibres may be pulp fibres from chemical pulp, e.g. kraft, sulphate or sulphite, mechanical pulp, thermo-mechanical pulp, chemo-mechanical pulp and/or chemo-thermo-mechanical pulp, abbreviated as CTMP. Pulps derived from both deciduous (hardwood) and coniferous (softwood) may be used. The fibres may hence be natural fibres from wood, for example, cellulose-based fibres, bamboo based fibres, and the like. Natural fibres can also be fibres which are not wood, such as cotton, abaca, kenaf, sabai grass, flax, esparto grass, straw, jute hemp, bagasse, milkweed floss fibres, and pineapple leaf fibres. The paper material can be made of one kind of fibres or a mix of different fibres. The paper material may also be treated in a way to stand moisture better, for example with a plastic layer on the inside or the outside. The paper may be a smooth paper which has been calendared. The paper may be a paper with low porosity. The paper material may include recycled or virgin fibres or a combination thereof.

Optionally, the sealed bottom of the bag is formed by folding and adhering a portion of the paper material to form a flat and rectangular bottom. The sealed bottom may advantageously be formed using overlap seams, providing a flat bottom. The bottom may preferably be a block bottom or a pinch bottom or any other kind of bottom.

The personal hygiene absorbent articles may be used for the absorption of bodily exudates, such as blood, urine, sweat and faeces. These personal hygiene absorbent articles may be packaged individually in wrappers, so that the article remains clean and intact prior to use. However, it is also possible to have two or three or more personal hygiene absorbent articles in a wrapper, which maybe suitable to bring along and use during the day. The wrapper may be a wrap where the article is folded together with wrapper material before the side edges, i.e. the edge areas, are sealed or a kind of pouch where the folded article is inserted into the pouch before the pouch is being sealed. Individual packages facilitate hygienic carrying of single articles for future use, e.g. in a handbag. Further, the packaging units are often used both as a means for packaging an unused article and for disposal of the used article.

Optionally, the at least two absorbent articles are arranged as at least one stack of absorbent articles. In this case, the absorbent articles may, e.g., be incontinent shields, sanitary napkins, panty liners, diapers, or the like. The at least one stack of absorbent articles may be at least one non-compressed stack of absorbent articles. Alternatively, the at least one stack of absorbent articles may be a compressed stack of absorbent articles. A stack of absorbent articles comprises at least two absorbent articles. The stack may, purely by way of example, and in particular when the bag comprises two stacks of articles, comprise 8 or 10 absorbent articles. However, the stack is not limited to 8 or 10 articles. Each stack may hence comprise more than 10 or less than 8 articles. When a single stack of absorbent articles is provided in the bag, the stack may comprise a larger number of articles, such as 15-25 absorbent articles, e.g., 16 or 20 absorbent articles.

Optionally, each one of the at least one stack of absorbent articles comprises a plurality of folded and individually wrapped absorbent articles. Alternatively, each one of the at least one stack of absorbent articles may comprise a plurality of folded absorbent articles without individual wraps. In both cases, the absorbent articles may be folded once or twice such that each folded absorbent article is folded into two or three folding layers. The fold or folds may preferably be arranged in parallel with the peripheral wall of the bag, i.e., perpendicularly to the bottom of the bag. Alternatively, the fold or folds may be arranged in parallel with the bottom of the bag.

The folded absorbent articles may have a width of 110-130 mm and a length of 70-95 mm.

Optionally, the at least one stack of absorbent articles comprises two stacks of absorbent articles. The two stacks of absorbent articles may be a bottom stack located in the bottom of the bag and a top stack located in the top of the bag. In some embodiments, the at least one stack of absorbent articles may comprise more than two stacks of absorbent articles, such as four stacks of absorbent articles. For example, two bottom stacks and two top stacks may be provided, wherein a first bottom stack and a first top stack are located closer to the first side wall, and wherein a second bottom stack and a second top stack are located closer to the second side wall.

Optionally, the at least one stack of absorbent articles comprises a single stack of absorbent articles.

Optionally, the bag is opaque and the absorbent articles, such as a wrapper of individually wrapped absorbent articles, or the absorbent articles, cannot be seen through the bag. Information printed on the package can thereby be made clearly visible to a user. Of course, the articles within the bag may be non-wrapped articles such as diapers or nonwrapped pads.

Optionally, the absorbent article is a disposable absorbent article such as a diaper, or an absorbent pad such as an incontinent shield or a sanitary napkin, or a washable and reusable absorbent article.

Further advantages and advantageous features of the invention are disclosed in the following description and in the dependent claims.

### BRIEF DESCRIPTION OF THE DRAWINGS

FIG. 1a is a perspective view of a package according to an embodiment in a first state,

FIG. 1b is a perspective view of the package in FIG. 1a in a second state,

FIG. 2 is a perspective view of the package in FIG. 1a without adhesive patch,

FIG. 3 is a top view of the package in FIG. 1b,

FIG. 4 is a top view of the package in FIG. 1a-b in an open state,

FIG. 5 is another perspective view of the package in FIG. 1b,

FIG. 6 is a side view of the package in FIG. 1b,

FIG. 7 schematically illustrates a folded and wrapped absorbent pad,

FIG. 8 illustrates the absorbent pad from FIG. 7,

FIG. 9 illustrates a paper sheet used to form a package according to an embodiment of the disclosure,

FIG. 10 illustrates a paper sheet used to form a package according to another embodiment of the disclosure,

FIG. 11 is a perspective view of a package according to a second embodiment,

FIG. 12 is a perspective view of a package according to a third embodiment,

FIG. 13 is a perspective view of a package according to a fourth embodiment,

FIG. 14 is a perspective view of a resealed package according to an embodiment, and

FIG. 15 is a perspective view of a package according to a fifth embodiment.

### DETAILED DESCRIPTION OF PREFERRED EMBODIMENTS

Various aspects of the disclosure will hereinafter be described in conjunction with the appended drawings to illustrate and not to limit the disclosure, wherein like designations denote like elements, and variations of the described aspects are not restricted to the specifically shown embodiments but are applicable on other variations of the disclosure.

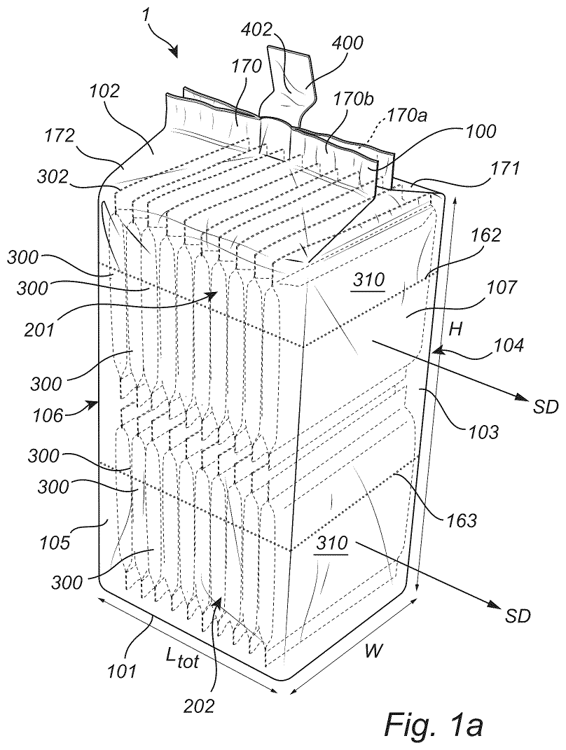

A package 1 according to embodiments of the disclosure is schematically illustrated in FIGS. 1-6. In the illustrated embodiments, the package 1 comprises a bag 100 and two stacks 201, 202 of absorbent articles 300 in the form of absorbent pads 300 arranged in the bag 100. A top stack 201 is arranged on top of a bottom stack 202. The bag 100 is made of a first paper material and has a sealed bottom 101, a top seal 170, and a peripheral wall 103 extending between the bottom 101 and the top seal 170. The peripheral wall 103 comprises a front wall 104, an opposite rear wall 105, and first and second side walls 106, 107 connecting the front wall 104 and the rear wall 105. The peripheral wall 103 further comprises a first top surface 171 extending between the top seal 170 and the front wall 104, and a second top surface 172 extending between the top seal 170 and the rear wall 105. The two top surfaces 171, 172 together with the top seal 170 form a sealed top 102 of the package 1. The top seal 170 comprises a first outer surface 170a facing forward, being in direct connection with the first top surface 171, and a second outer surface 170b facing rearward, being in direct connection with the second top surface 172.

The package 1 further comprises at least one adhesive patch 400 configured to hold the top seal 170 folded onto one of the first and the second top surfaces 171, 172. In the embodiment illustrated in FIGS. 1a-b, a single adhesive patch 400 is provided, which comprises a non-adhesive front surface 401 and a back surface 402. An adhesive layer is provided on the back surface 402. FIG. 1a illustrates the package 1 with the top seal 170 in a sealed but non-folded state, with the adhesive patch 400 attached only to the first outer surface 170a of the top seal 170. FIG. 1b illustrates the package 1 with the top seal 170 in a sealed and folded state, with a first portion 403 of the adhesive patch 400 adhered to the first outer surface 170a of the top seal 170, and with a second portion 404 adhered to the second top surface 172 onto which the top seal 170 is folded, and further reaching onto and being adhered to the rear wall 105. Of course, the top seal 170 may instead be folded onto the first top surface 171, in which case the first portion 403 would be adhered to the second outer surface 170b of the top seal 170 and the second portion 404 of the adhesive patch 400 would be adhered to the first top surface 171 and to the front wall 104. In some embodiments, the adhesive patch 400 may have another shape and may not reach all the way onto the front wall 104 or the rear wall 105. Furthermore, in some embodiments, the first portion 403 of the adhesive patch 400 may reach onto the top surface 171 connected to the outer surface of the top seal 170 onto which the first portion 403 of the adhesive patch 400 is adhered, and optionally all the way onto the one of the rear wall 105 and the front wall 104 connected thereto.

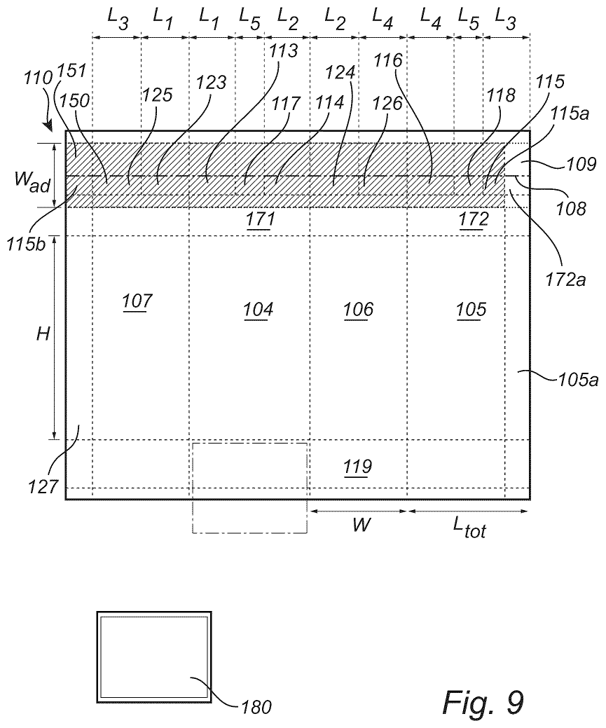

The bag 100 is formed from a single sheet 110 of rectangular paper as illustrated in FIG. 9, which is folded, glued, and cut to form the bag 100. Once the bottom 101 and the peripheral wall 103 of the bag 100 have been formed and sealed, and the bag 100 has been filled with the absorbent pads 303, the bag 100 is folded and sealed at the top 102 to form the top seal 170 and a superfluous top portion 109 above a cutting line 108 as illustrated in FIG. 9 is cut away.

The top seal 170 has been formed by folding and adhering inner surface top portions of the paper sheet 110 to one another so as to form wings, or fins. FIG. 9 illustrates the rectangular sheet 110 with a first side facing outwards, wherein the first side will form an exterior side of the bag 100 after folding and sealing. The inner surface top portions are provided on a second side of the sheet 110, which will after folding and sealing form an interior side of the bag 100. Locations of the inner surface top portions, although provided on the second side, are indicated in FIG. 9. The inner surface top portions comprise: a first top portion 113 of the first top surface 171 and an adjacent first top portion 123 of the second side wall 107; a second top portion 114 of the first top surface 171 and an adjacent first top portion 124 of the first side wall 106; a first top portion 115 of the second top surface 172 and an adjacent second top portion 125 of the second side wall 107; a second top portion 116 of the second top surface 172 and an adjacent second top portion 126 of the first side wall 106; a third top portion 117 of the front wall 104, located between the first and second top portions 113, 114 of the first top surface 171; and a third top portion 118 of the second top surface 172, located between the first and second top portions 115, 116 of the second top surface 172.

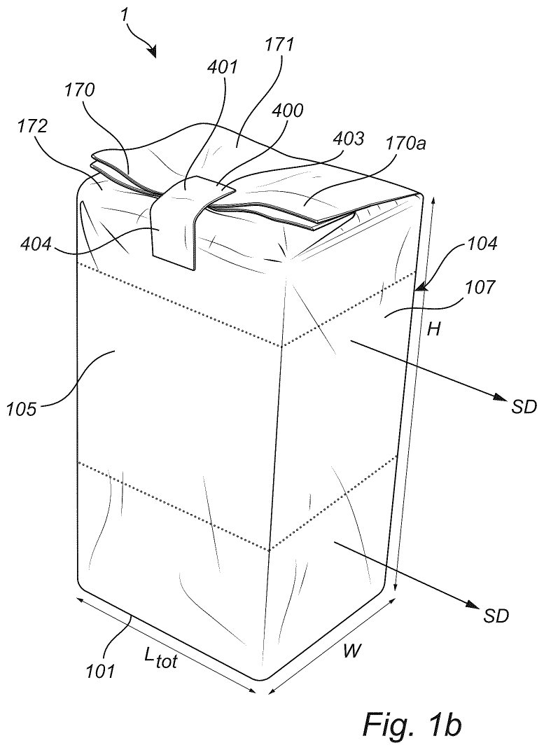

The first top portion 113 of the first top surface 171 is adhered to the adjacent first top portion 123 of the second side wall 107 to form a first adhered portion 111 having a first length L₁ as shown in FIG. 2 and FIG. 9. The second top portion 114 of the first top surface 171 is adhered to the adjacent first top portion 124 of the first side wall 106 to form a second adhered portion 112 having a second length L₂. The first top portion 115 of the second top surface 172 is adhered to the adjacent second top portion 125 of the second side wall 107 to form a third adhered portion 121 having a third length L₃. The second top portion 116 of the second top surface 172 is adhered to the adjacent second top portion 126 of the first side wall 106 to form a fourth adhered portion 122 having a fourth length L₄. The third top portion 117 of the first top surface 171 is adhered to the third top portion 118 of the second top surface 172 to form a fifth adhered portion 130 having a fifth length L₅. The fifth length L₅ is smaller than each one of the first length L₁, the second length L₂, the third length L₃, and the fourth length L₄. The lengths L₁, L₂, L₃, L₄ and L₅ are all measured in a direction parallel with the front wall 104, with the adhered portions 111, 112, 121, 122, 130 stretched out to be as long as possible.

In the illustrated embodiments, the first length L₁, the second length L₂, the third length L₃, and the fourth length L₄ are all equal, within manufacturing tolerances. The fifth length L₅ is herein less than less than 75% of each one of the first length L₁, the second length L₂, the third length L₃, and the fourth length L₄, preferably less than 65%, or less than 60%. The first length L₁, the second length L₂, the third length L₃, and the fourth length L₄ may be between 30-50 mm, preferably 35-45 mm. The fifth length L₅ may be 1-30 mm, more preferably 3-25 mm. A total length Lₜₒₜ of the front wall 104 and/or back wall 105 of the bag 100, as measured between the side walls 106, 107 at a bottom of the package 1, is essentially equal to the sum of the first length L₁, the second length L₂, and the fifth length L₅ and/or the sum of the third length L₃, the fourth length L₄ and the fifth length L₅.

In the illustrated embodiment, the first length L₁ is 39 mm and the fifth length L₅ is 22 mm, i.e., 56% of the first length L₁. In the illustrated embodiment the bag 100 has a total length Lₜₒₜ of 100 mm as measured between the side walls 106, 107, and a total width W as measured between the front and rear walls 104, 105 of 78 mm. A filling height H of the bag 100 is in the illustrated embodiment 163 mm.

In another embodiment, the first length L₁ is 45 mm and the fifth length L₅ is 10 mm, i.e., 22% of first length L₁. The bag 100 has a total length Lₜₒₜ of 100 mm as measured between the side walls 106, 107, and a total width W as measured between the front and rear walls 104, 105 of 88 mm. A filling height H of the bag 100 is in the illustrated embodiment 165 mm.

As best seen in FIG. 2, the fifth adhered portion 130 connects the first and third adhered portions 111, 121, provided closer to the second side wall 107, to the second and fourth adhered portions 112, 122, provided closer to the first side wall 106. The first and third adhered portions 111, 121 hence form a first pair of wings and the second and fourth adhered portions 112, 122 forms a second pair of wings. None of the adhered portions 111, 112, 121, 122, 130 is adhered to another one of the adhered portions 111, 112, 121, 122, 130 by e.g. an overlap seam or a butt seam. Furthermore, none of the adhered portions 111, 112, 121, 122, 130 is directly adhered to an outer surface portion of the peripheral wall 103. Since they are not adhered to any outer surface portion of the peripheral wall 103, the first, second, third and fourth adhered portions 111, 112, 121, 122, form suitable gripping members for opening of the bag.

In alternative embodiments, the first and third adhered portions 111, 121 may be adhered to one another, and/or the second and fourth adhered portions 112, 122 may be adhered to one another.

In the illustrated embodiments of FIGS. 1-6, the adhesive patch 400 is positioned on the top seal 170 half-way between the first and second side walls 106, 107 of the bag 100. In other words, it is adhered onto the fifth adhered portion 130 illustrated in FIG. 2. For clarity, the fifth adhered portion 130 is in FIG. 2 illustrated without the adhesive patch 400 adhered thereto.

The fifth portion 130 may form a weak link of the top seal 170, which will be further described below with reference to FIG. 9, and the adhesive patch 400 placed thereon is therefore useful to ensure that the package 1 is properly sealed.

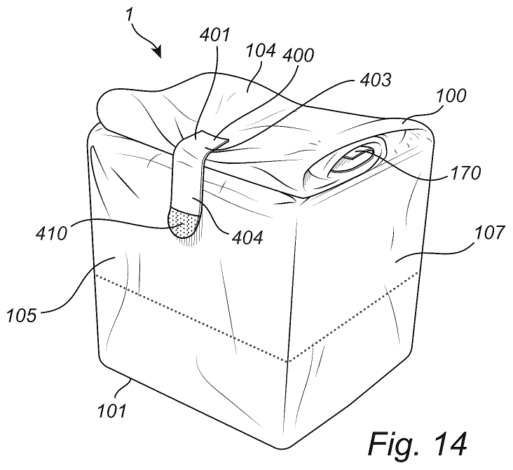

The adhesive layer of the adhesive patch 400 may be a self-adhesive layer, such as a self-adhesive layer configured to allow resealing of the bag 100 by the adhesive patch 400. A self-adhesive layer is an adhesive layer which sticks without wetting. FIG. 14 illustrates a package 1 such as the package 1 illustrated in FIGS. 1-6 in an opened, folded, and resealed state, wherein the adhesive patch 400 is used for resealing of the package 1. The adhesive patch 400 herein comprises a non-adhesive gripping portion 410 configured to be gripped by a user for releasing at least the second portion 404 of the adhesive patch 400 from the bag 100. The gripping portion 410 is located adjacent to the second portion 404 of the adhesive patch 400.

The adhesive patch 400 comprises a backing material on which the adhesive layer is provided. The backing material may be any type of tape backing materials well known in the art. For example, paper materials, polyester films, polypropylene films, paper backings, or other materials may be used as the backing material.

In the embodiment illustrated in FIG. 14, the user has removed all articles 300 of the top stack 201 and rolled down the bag 100 before using the adhesive patch 400 to reseal it. The entire adhesive patch 400 has been removed from the bag 100 in connection with rolling of the bag 100, and subsequently re-attached with the first portion 403 adhered to a portion of the bag 100 which in the sealed state constituted the front wall 104, and with the second portion 404 adhered to the rear wall 105. Herein, the same adhesive may be used for the adhesive layer in the first and second portions 403, 404 of the adhesive patch 400, allowing releasing and resealing of the entire adhesive patch 400.

In alternative embodiments, a different adhesive may be used for the first portion 403 than for the second portion 404. The first portion 403 may comprise an adhesive that securely attaches the adhesive patch 400 to the top seal 170 of the bag 100. The second portion 404 may comprise an adhesive that forms a releasable bond with another surface of the bag 100. In such embodiments, resealing of the opened bag 100 may be achieved by folding down an upper part of the bag 100, including the broken top seal 170, and attaching the second portion 404 of the adhesive patch 400 to, e.g., the front wall 104 or the rear wall 105. The first portion 403 of the adhesive patch 400 remains securely attached to the broken top seal 170 during the resealing.

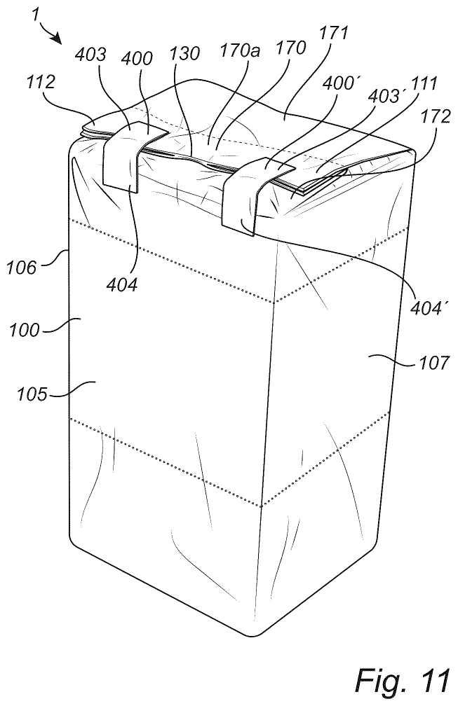

FIG. 11 illustrates a second embodiment of the package 1, which only differs from the first embodiment illustrated in FIGS. 1-6 in that a first adhesive patch 400 and a second adhesive patch 400′ are provided instead of a single adhesive patch. The patches 400, 400′ are together configured to hold the top seal 170 folded onto the one of the first and the second top surfaces 171, 172, herein to the second top surface 172. The two adhesive patches 400, 400′ are herein identical, although they may in other embodiments have different shapes or differ in other ways. The first adhesive patch 400 has a first portion 403 attached to the first outer surface 170a of the top seal 170 close to the first side wall 106, i.e., to the second adhered portion 112, between the fifth adhered portion 130 and the first side wall 106. The second adhesive patch 400′ has a first portion 403′ attached to the first outer surface 170a of the top seal 170 close to the second side wall 107, i.e., to the first adhered portion 111, between the fifth adhered portion 130 and the second side wall 107. Both adhesive patches 400, 400′ have second portions 404, 404′ adhered to the second top surface 172 and to the rear wall 105 connected thereto.

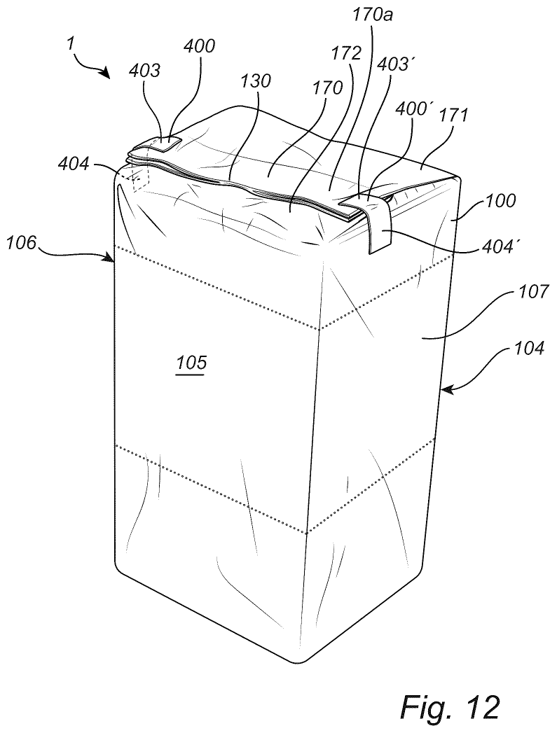

FIG. 12 illustrates a third embodiment of the package 1, which only differs from the second embodiment in how the first and second adhesive patches 400, 400′ are adhered to the bag 100. In the third embodiment, the first portion 403 of the first adhesive patch 400 is adhered on the one hand to the first outer surface 170a of the top seal 170, and the second portion 404 is adhered to the first side wall 106. The first portion 403′ of the second adhesive patch 400′ is adhered to the first outer surface 170a of the top seal 170, and the second portion 404′ is adhered to the second side wall 107. In this embodiment, the adhesive patches 400, 400′ are not adhered to any one of the front and rear walls 104, 105.

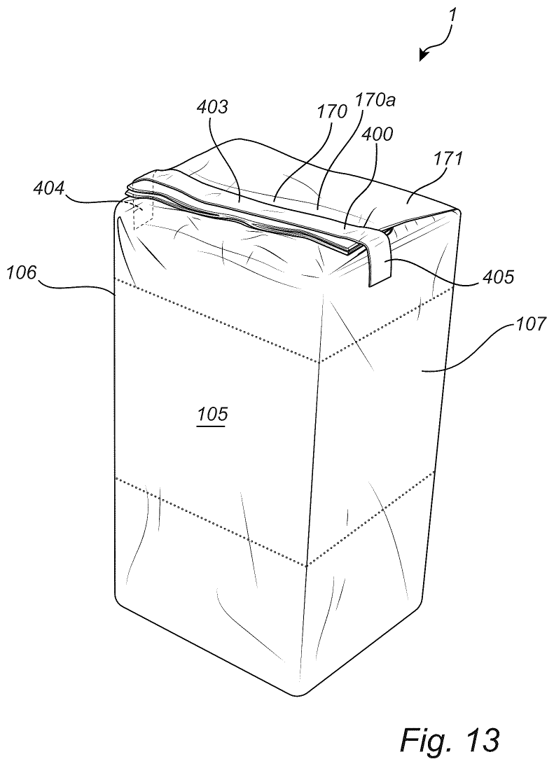

FIG. 13 illustrates a fourth embodiment of the package 1, which only differs from the third embodiment in that instead of two adhesive patches 400, 400′ adhered to opposite side walls 106, 107, a single adhesive patch 400 is provided which extends from the first side wall 106 all the way to the second side wall 107. The adhesive patch 400 has a first portion 403 adhered to the first outer surface 170a of the top seal 170, a second portion 404 adhered to the first side wall 106, and a third portion 405 adhered to the second side wall 107. It hence has an elongated shape and may be referred to as an adhesive strip.

In any one of the embodiments illustrated in FIGS. 11-13, each adhesive patch 400, 400′ may of course be provided with at least one non-adhesive gripping portion as described with reference to FIG. 14.

In the illustrated embodiments, the sealed top 102 of the package 1 forms a planar or an essentially planar surface extending in parallel with the sealed bottom 101 when the adhesive patch 400 or patches 400, 400′ hold/s the top seal 170 folded onto one of the first and the second top surfaces 171, 172. Hence, the package 1, when in the sealed and folded state shown in e.g. FIG. 1b, has the basic shape of a cuboid (rectangular block).

The bag 100 comprises an adhesion layer 150 provided on the inner surface portions 113, 114, 115, 116, 117, 118, 123, 124, 125, 126 of the peripheral wall 103, as shown in FIG. 9, wherein the adhesion layer 150 is shown as a shaded area. The adhesion layer 150 is provided on the second side of the sheet 110, forming the interior of the bag 110. The adhesion layer 150 is used for adhering the inner surface portions to one another to form the butt seams as described above. The adhesion layer may have a basis weight of 28-45 g/m², preferably 30-40 g/m², or more preferably 34-38 g/m² and be a hot-melt adhesion layer.

The basis weight of the adhesion layer 150 may be determined by weighing a first sample of the sheet 110 on which the adhesion layer 150 is provided, and a second sample of the same size on which no adhesion layer is provided. Alternatively, the adhesion layer may be removed from the first sample, whereafter the first sample is weighed again. A total weight of the adhesion layer provided on the first sample is thereby obtained as the difference in weight between the samples with and without adhesion layer. The weight of the adhesion layer is divided by a surface area of the first sample to obtain the basis weight.

The adhesion layer 150 can, for example, comprise or consist of a hot melt adhesive. However, the adhesive is not limited to a hot melt adhesive, it may be any kind of adhesive suitable for the application. Other kind of adhesives can be adhesives which can dissolve in water. Such adhesives include starch based adhesives, polyvinyl alcohol based adhesives, and polyethylene oxide based adhesives. If the adhesive is not water-soluble, then water-dispersible adhesives may similarly be utilized. Suitable examples of water dispersible adhesives include thermoplastic elastomer based adhesives and polyvinyl acetate based adhesives. Any suitable pressure sensitive adhesives may also be used.

The adhesion layer 150 is herein partially provided on the superfluous top portion 109 which is cut away after sealing. The adhesion layer 150 may prior to adhesion, folding and cutting have a width W_{ad} of 30-60 mm as measured in a height direction of the bag 100.

The top seal 170 at the top 102 of the bag 100 can be accomplished by pressing two seal bars against each other, with the folded top portions 113, 114, 115, 116, 117, 118, 123, 124, 125, 126 of the bag 100 comprising the adhesion layer 150 inserted between the seal bars. Thereby, the hot melt adhesive applied to the inside of the bag 100 melts and the folded top portions 113, 114, 115, 116, 117, 118, 123, 124, 125, 126 attach to each other and form the top seal 170. The sealing width accomplished by the sealing bars may be 3-10 mm measured in a height direction of the bag 100, more preferably 3-6 mm. After the bag 100 has been cut, the sealing width may be smaller or the same, depending on if the cutting line 108 along which the cut is made is above the seal or not.

The seal of the packages may have a tensile strength of at least 1 N per 25.4 mm. For example, each of the plurality of seals of the packages of the present disclosure may have a tensile strength of between 1 N per 25.4 mm to about 22 N per 25.4 mm, more preferably from about 3 N per 25.4 mm to about 17 N per 25.4 mm. The tensile strengths of the seals mentioned herein can be determined by the tensile test method described in ASTM F88-21 as modified herein.

As mentioned above, the fifth portion 130 may form a weak link of the top seal 170. This can be due to the uneven thickness of the top seal 170 when the inner surface portions of the peripheral wall are sealed to one another after filling the bag 100 with the absorbent articles 300. As described, the top seal 170 can be accomplished by pressing two seal bars against each other, with the folded top portions 113, 114, 115, 116, 117, 118, 123, 124, 125, 126 of the bag 100 comprising the adhesion layer 150 inserted between the seal bars. Thereby, the hot melt adhesive applied to the inside of the bag 100 melts and the folded top portions 113, 114, 115, 116, 117, 118, 123, 124, 125, 126 attach to each other and form the top seal 170. Due to an uneven thickness, arising since the fifth portion 130 only comprises two layers of material compared to four layers of material at the first and third adhered portions 111, 121 and at the second and fourth adhered portions 112, 122, it might be the case that the heat and/or pressure applied in the area of the fifth portion 130 is/are insufficient, causing the two layers forming the fifth portion 130 to not stick together properly.

As used herein, a paper material is to be understood as a material produced by pressing together moist fibres, dewatering, and drying the fibres into preferably flexible sheets. The paper material used herein may have a basis weight between 50 and 90 g/m² as measured according to the ISO 536 standard. A ratio between the basis weight of the paper material used for the bag 100 and the basis weight of the adhesion layer 150 may be within a range of 1.6-2.5, preferably 1.8-2.2. For example, the paper material may have a basis weight of 70 g/m² and the adhesion layer 150, such as the hot-melt adhesion layer, may have a basis weight of approximately 36 g/m².

The first paper material may have any one or more of following properties:
a tensile strength measured according to the ISO 1924-3 standard in a machine direction between 4 and 10 kN/m, preferably between 6 and 8 kN/m, and in a cross direction between 2 and 6 kN/m, preferably between 3 and 5 kN/m;
a tear index measured according to the ISO 1974 standard in a machine direction between 8 and 14 mNm²/g, and in a cross direction between 8 and 15 mNm²/g;
a tear strength measured according to the ISO 1974 standard in a machine direction between 400 and 1300 mN, and in a cross direction between 450 and 1400 mN,
a stretch measured according to the ISO 1924-3 standard in a machine direction between 1 and 6 %, and in a cross direction between 5 and 12 %.

The second paper material may have the same properties or differ.

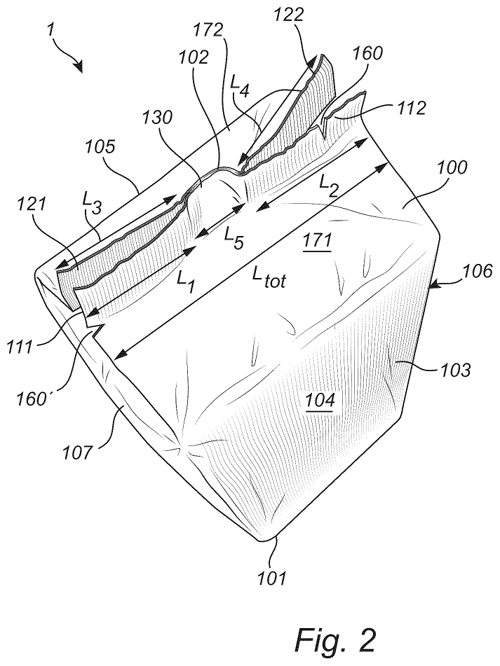

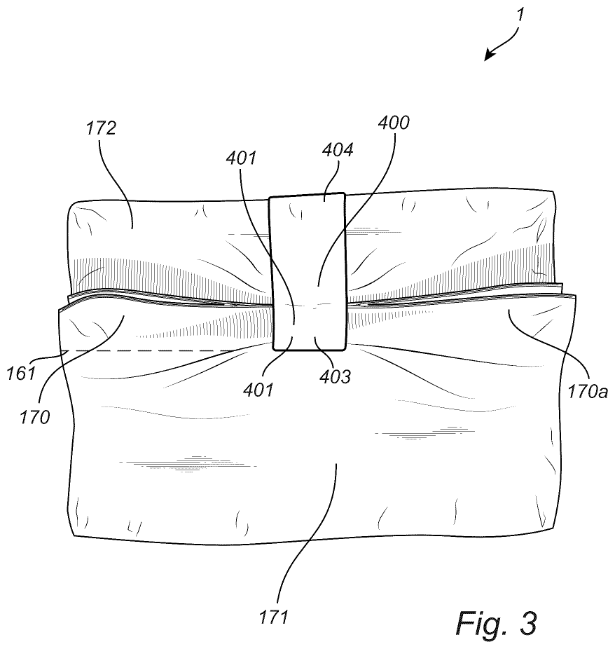

As illustrated in FIG. 2, the bag 100 may in certain embodiments comprise a first notch 160 for tearing open the bag 100. The first notch 160 is herein formed at a top edge of the second adhered portion 112, although it may alternatively be provided in any one of the first, third and fourth adhered portions 111, 121, 122. As further illustrated, the bag 100 may alternatively or additionally comprise a second notch 160′ for tearing open the bag 100, the second notch 160′ being formed just below one of the adhered portions, herein below the first adhered portion 111. In alternative embodiments, the notches 160, 160′ may be replaced or complemented by a first weakening line 161 in the form of a perforation line or a score line such as illustrated in FIG. 3. The first weakening line may e.g. be located below the top seal 170 such as illustrated in FIG. 3. In yet other embodiments, no notch or first weakening line is provided. In those cases, the bag 100 may be opened by, e.g. manually pulling on opposite adhered portions. The package 1 in an open state is illustrated in FIG. 4.

The package 1 may further comprise one or more second weakening lines 162, 163 configured for removing a portion of the bag 100 in connection with usage. In the first embodiment illustrated in FIGS. 1-6, an upper second weakening line 162 and a lower second weakening line 163 are provided, both extending around the peripheral wall 103. The upper second weakening line 162 extends at a level such that when the user tares the bag along the upper second weakening line 162, the articles 300 within the top stack 201 are exposed and easily accessible for the user. The lower second weakening line 163 extends at a level such that, when the bag 100 is tared along the lower second weakening line 163, the articles 300 within the bottom stack 202 are exposed and easily accessible for the user.

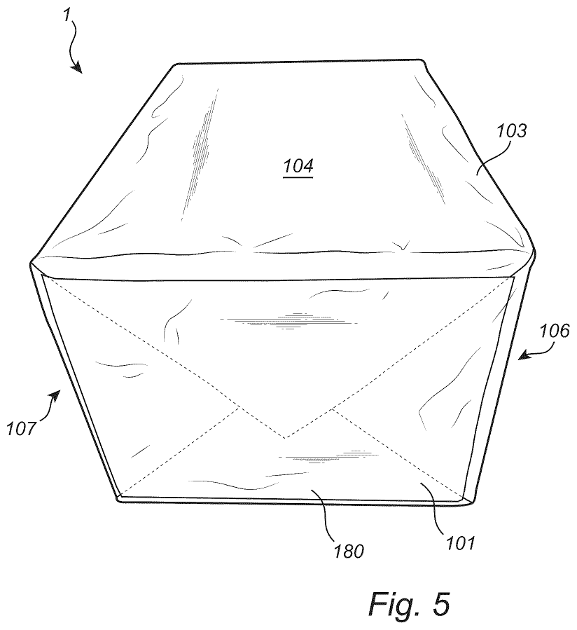

The rectangular sheet 110 is in FIG. 9 illustrated with dashed folding lines along which the sheet 109 is folded to form the bag 100. As mentioned earlier, the rectangular sheet 110 is illustrated from its first side, forming the exterior wall of the bag 100 after folding and sealing. To form the peripheral side wall 103, an edge portion 105a of the rear wall 105 is overlapped with a rectangular seam portion 127 located next to the second side wall 107 on the rectangular sheet 110. After folding the sheet 110, the edge portion 105a of the rear wall 105 overlaps the seam portion 127 and an overlap seam 140 is formed. Hence, the seam portion 127 is hidden behind the edge portion 105a and forms part of the rear wall 105 after folding and sealing. The overlap seam 140 extends from the bottom 101 of the bag 100 to the top 102 of the bag 100 as illustrated in FIG. 6. An adhesive (not shown) may be used to form the overlap seam 140. The adhesive for forming the overlap seam 140 may be arranged as a layer on an interior side of the edge portion 105a, but preferably the adhesive is arranged as a layer on the seam portion 127, i.e., on the first side of the rectangular sheet 110 which after folding and sealing faces outwards. Hence, the adhesive layer for forming the overlap seam 140 and the adhesion layer 150 are arranged on opposite sides of the sheet 110. The adhesive for forming the overlap seam 140 may be of the same type as used for the adhesion layer 150, or of a different type. The adhesive may, e.g., be a hot-melt adhesive, i.e., a thermoplastic adhesive which is not water-soluble. However, the adhesive is not limited to a hot melt adhesive, it may be any kind of adhesive suitable for the application. Other kind of suitable adhesives can be adhesives which can dissolve in water. Such adhesives include starch based adhesives, polyvinyl alcohol based adhesives, and polyethylene oxide based adhesives. If the adhesive is not water-soluble, then water-dispersible adhesives may similarly be utilized. Suitable examples of water dispersible adhesives include thermoplastic elastomer based adhesives and polyvinyl acetate based adhesives. Any suitable pressure sensitive adhesives may also be used.

The seam portion 127 illustrated in FIG. 9 comprises an end portion 151 of the adhesion layer 150, provided on an inner surface portion 115b which forms part of the first top portion 115 of the second top surface 172 after the overlap seam 140 has been formed. The inner surface portion 115b together with an adjacent inner surface portion 115a hence forms the first top portion 115 of the second top surface 172. The inner surface portion 115b is located inside of an edge portion 172a of the second top surface 172 after the overlap seam 140 has been formed, i.e., on the inside of the package 1. The adhesion layer 150 does not extend onto the edge portion 172a of the second top surface 172. The edge portion 105a of the rear wall 105 and the edge portion 172a of the second top surface 172 may herein have a width of about 25 mm.

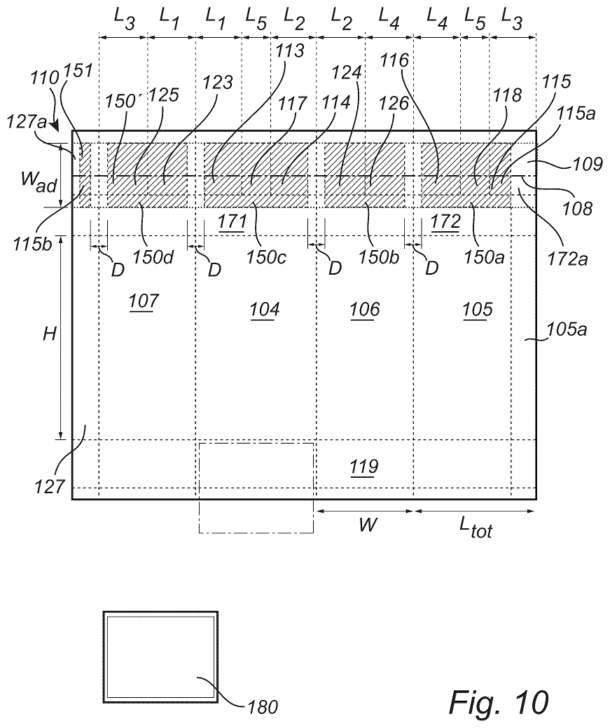

The adhesion layer 150 shown in FIG. 9 is a continuous adhesion layer. However, an alternative is shown in FIG. 10, wherein the adhesion layer 150′ is a discontinuous adhesion layer comprising a first layer portion 150a provided on the second top surface 172, a second layer portion 150b provided on the first side wall 106, a third layer portion 150c provided on the first top surface 171, a fourth layer portion 150d provided on the second side wall 107, and an end layer portion 151 provided on the seam portion 127. Between each two adjacent layer portions 150a-d, 151, an area free of adhesive is provided. The dashed folding lines are located in the areas free of adhesive. A distance D between each two adjacent layer portions 150a-d, 151 may be in a range of 5-15 mm, preferably 8.5-11.5 mm, such as about 10 mm. This prevents the corners from becoming adhered during manufacturing of the bag 100, prior to filling it with the absorbent pads 300. In the embodiment illustrated in FIG. 10, the discontinuous adhesion layer 150′ does not extend onto the edge portion 105a of the rear wall 105 nor onto an edge portion 127a of the seam portion 127. The adhesive free edge portion 127a of the seam portion 127 may herein have a width of about 5 mm. The overlap seam 140 may be formed in the same way as described above with reference to FIG. 9, such as by providing an adhesive layer on the seam portion 127, i.e., on the first side of the rectangular sheet 110 which after folding and sealing faces outwards. Similarly to FIG. 9, FIG. 10 illustrates the sheet 110 from its first side, forming the exterior wall of the bag 100 after folding and sealing.

The bag 100 may further comprise an air displacement hole (not shown) located at or close to the bottom 101 of the bag 100 for allowing air to escape from the bag 100, such as when loading the pads 300 into the bag 100 and for reducing tension that may arise as the bag 100 is subjected to pressure. The air displacement hole may be located close to an edge between any two adjacent walls. It may preferably be located at one of the side walls 106, 107, adjacent to the bottom 101. It may alternatively be formed as a non-sealed lower portion of the overlap seam 140.

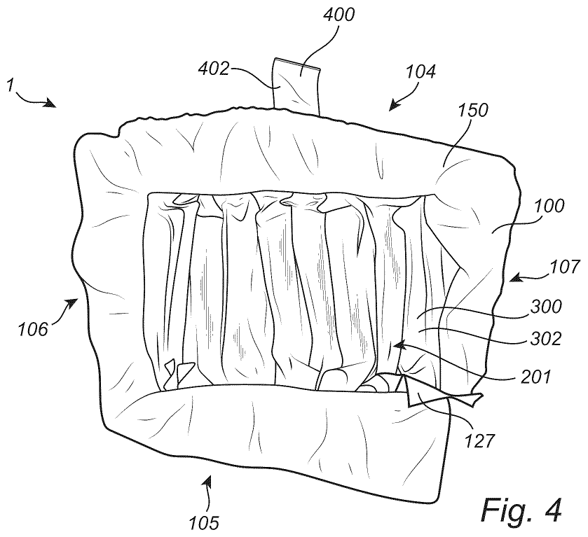

An example configuration of the bottom 101 is illustrated in FIG. 5. The sealed bottom 101 of the bag 100 is a block bottom, herein formed by folding a rectangular lower portion 119 of the sheet 110, and adhering the resulting folded portions to each other to form a flat and rectangular bottom 101. The sealed bottom 101 is formed using overlap seams to provide a flat bottom. A rectangular patch 180, forming a bottom label, is glued as a cover onto the bottom 101.

All external sides of the bag 100 may be covered with a layer of varnish, protecting any printed portions of the bag 100. Printed portions may be provided on the peripheral wall 103 as well as on the top 102 and/or on the bottom label.

Since the absorbent pads 300 are non-compressed or only slightly compressed, the package 1 has relatively sharp 90° edges. The front and rear walls 104, 105 as well as the first and second side walls 106, 107 are relatively flat. The length Lₜₒₜ of the package 1 as well as the width W are therefore close to constant along the height H of the package, as can be gleaned from FIG. 1. A maximum total length Lₜₒₜ of the package 1, as measured anywhere along the stacking height H, preferably differs by 10 mm or less, more preferably by 8 mm or less, from a minimum total length Lₜₒₜ as measured at the sealed bottom 101 or at the sealed top 102. In other words, the side walls 106, 107 do not bulge out significantly.

In other examples of the package 1, the absorbent articles 300 may be diapers, such as diapers arranged in one or two stacks within the bag 100. Diapers are often significantly compressed within the bag 100, and this may cause the side walls 106, 107 to bulge out. The maximum total length Lₜₒₜ of the package 1 may in those cases differ by 10 mm or more from the minimum total length Lₜₒₜ measured at the sealed bottom 101 or at the sealed top 102.

Two stacks 201, 202 of folded and individually wrapped absorbent pads 300, such as disposable personal hygiene absorbent pads in the form of incontinent shields, sanitary napkins, panty liners, or the like, are in most of the illustrated embodiments arranged in the bag 100.

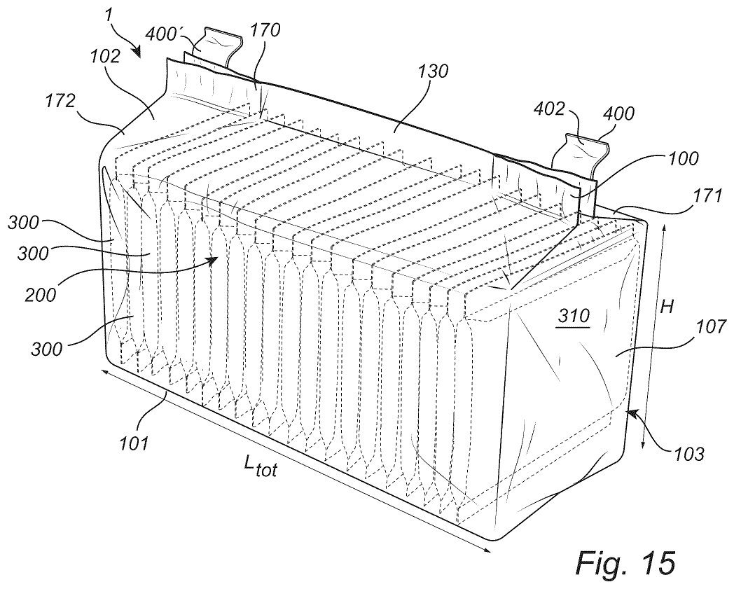

In a fifth embodiment illustrated in FIG. 15, the package 1 comprises a single stack 200 of folded and individually wrapped absorbent pads 300. The single stack 200 herein comprises a larger number of folded and optionally individually wrapped pads 300 than the package 1 illustrated in FIG. 1a. The package 1 according to the fifth embodiment hence has a smaller height H and a larger length Lₜₒₜ than the package 1 according to the first embodiment. In particular, the fifth adhered portion 130 is longer than in the first embodiment. The bag 100 comprises two adhesive patches 400, 400′ for holding down the top seal 170. In FIG. 15, the package 1 is standing on the closed bottom 101. However, the package 1 can be arranged so that it stands on its first or second side wall 106, 107, for example, in a shop or on a shelf. The top seal 170 will then be arranged vertically on a shelf instead of horizontal. Alternatively, the package 1 can be arranged so that it stands on the closed top 102 comprising the top seal 170.

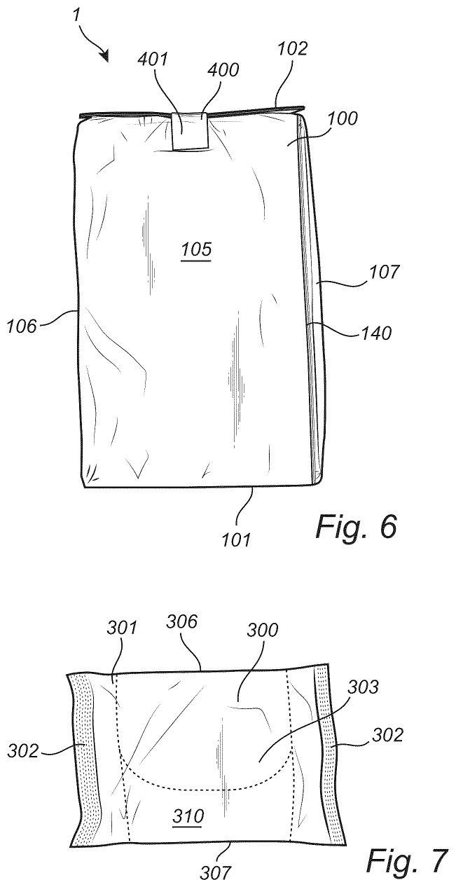

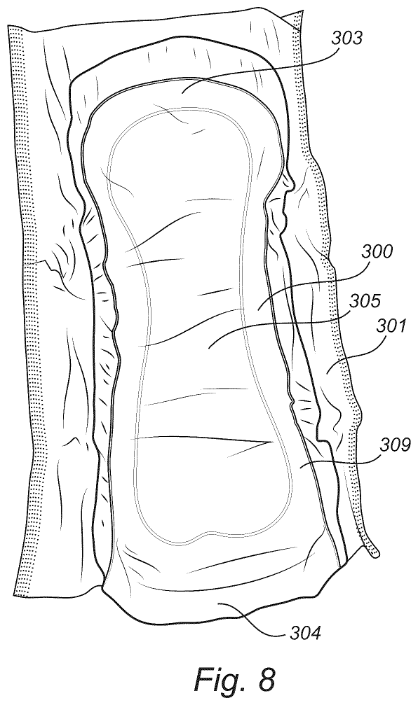

FIGS. 7-8 illustrate an absorbent pad 300 in the form of an incontinent shield according to an example embodiment. Such incontinent shields are, e.g., described in WO2017086849. The absorbent pad 300 may, purely by way of example, comprise a liquid pervious topsheet 309 bonded to an underlying acquisition or distribution layer (not shown), a liquid impermeable backsheet (not shown), and an absorbent core (not shown) sandwiched between the acquisition or distribution layer and the liquid impermeable backsheet. However, many different configurations are possible.

The illustrated absorbent pad 300 is a wing-less incontinent shield that is intended to be adhesively attached to an underwear of a user, for example by means of adhesive that is provided on an outer side of the backsheet for fastening the absorbent pad 300 to said underwear. However, the disclosure is equally applicable to wing-type absorbent pads, where laterally protruding wings are intended to be folded around the edge of the underwear of a user for fastening the absorbent pad 300 to said underwear.

The liquid permeable topsheet 309 can comprise a nonwoven material, e.g. spunbonded, meltblown, carded, hydroentangled, wetlaid, etc. which can be composed of natural fibres, such as woodpulp or cotton fibres, synthetic fibres, such as polyester, polyethylene, polypropylene, viscose, etc. or from a mixture thereof. The topsheet 309 material may further be composed of tow fibres, porous foams, apertured plastic films etc. The materials suited as topsheet materials should be soft and non-irritating to the skin and be readily penetrated by body fluid, e g urine or menstrual fluid, and display low rewetting properties. The topsheet 309 may comprise weldable fibres for enabling bonding to the underlying sheet by means of welding, such as ultrasonic welding. Alternatively, the topsheet 309 may be bonded to the underlying material with adhesive, or just placed on top of the underlying material.

The acquisition or distribution layer, if such a layer is used in absorbent pad 300, may comprise a material suitable for bonding to the topsheet 309. For example, if welding technology is used for bonding the topsheet 309 to the acquisition or distribution layer, the material of the acquisition or distribution layer should include at least partly weldable fibres. For example, the weldable fibres may polyethylene fibres coated with a layer of polypropylene. The acquisition or distribution layer may further include pure polyethylene fibres. In a specific example less than 30% of the fibres in the acquisition or distribution layer are polyethylene fibres and more than 70% of the fibres are welding fibres consisting of polyethylene fibres coated with polypropylene.

The absorbent core may comprise any conventional material suitable for absorbing discharged bodily wastes, such as cellulosic fluff pulp, tissue layers, highly absorbent polymers (superabsorbents), absorbent foam materials including hydrogel-foam material, absorbent nonwoven materials or the like. Similar to above, if welding technology is used for bonding the topsheet 309 directly to the absorbent core, the material of the absorbent core should include at least partly weldable fibres.

The liquid impermeable backsheet may comprise a thin plastic film, e.g. a polyethylene or polypropylene film, a nonwoven material coated with a liquid impervious material, a hydrophobic nonwoven material which resists liquid penetration or laminates of plastic films and nonwoven materials. The backsheet material may be breathable so as to allow vapour to escape from the absorbent core, while still preventing liquids from passing through the backsheet material.

In one embodiment, the absorbent pad 300 may be an absorbent pad sold commercially by Essity Hygiene and Health AB under the name TENA discreet Mini. The package 1 may then comprise two stacks 201, 202 with, purely by way of example, 8 or 10 absorbent pads 300 in each stack 201, 202.

In another embodiment, the absorbent pad 300 may be an absorbent pad sold commercially by Essity Hygiene and Health AB under the name TENA discreet Mini Plus.

The package 1 may then comprise two stacks 201, 202 with, purely by way of example, 8 or 10 absorbent pads 300 in each stack 201, 202.

Each absorbent pad 300 is in the illustrated embodiment provided within an individual wrapper 301, such as a thin sheet or similar, to which the absorbent pad 300 is releasably adhered before use. The sheet material for forming the wrapper 301 may be a single ply sheet of any suitable material known to the person skilled in the art, such as polyethylene or polypropylene film or nonwoven or paper. The wrapper 301 may also be a laminate comprising at least two distinct layers. Laminates suitable for packaging of hygiene articles are assumed to be known to the person skilled in the art and are not in any way limiting for the present disclosure.

The absorbent pad 300 attached to the individual wrapper 301 is herein folded twice to form three folding layers, with a front portion 303 overlapping a rear portion 304, both the front and rear portions 303, 304 overlapping a centre portion 305. In the embodiment illustrated in FIG. 8, the pad 300 is folded together with the wrapper 301. Alternatively the pad may be pre-folded and inserted into the wrapper. Each individually wrapped absorbent pad 300 comprises two sealed wrapper portions 302 extending perpendicularly to opposite folded edges 306, 307 of the absorbent pad 300, see FIG. 7. One of the sealed wrapper portions 302 faces upward within the bag 100 as illustrated in FIG. 1 and FIG. 4. Hence, a user may easily remove a wrapped absorbent pad 300 from the opened top of the bag 100 by grabbing the sealed wrapper portion 302. However, the sealed wrapper portion 302 is not limited to be arranged facing upward with in the bag 100. It may alternatively face any one of the other surfaces of the bag 100.

The sealed wrap portions 302 may be sealed by an adhesive, for example a resealable adhesive or a non-resealable adhesive and/or sealed by means of ultrasonic welding or heat welding. By the term “resealable adhesive” is meant an adhesive that provides a non-permanent adhesive bond between two adherent surfaces, i.e. a bond that may be broken by applying a pulling force to the adherent, and recreated by applying a pressing force to the adherent. The resealable adhesive may be a pressure-sensitive hotmelt adhesive.

A stacking direction SD of each stack 201, 202 within the bag 100 is perpendicular to the first and second side walls 106, 107 of the bag 100, see FIG. 1. Hence, within each stack 201, 202, the folded and individually wrapped absorbent pads 300 are stacked with side surfaces 310 thereof facing one another. The side surfaces 310 are aligned with the first and second side walls 106, 107 of the bag 100. A further, alternative, way of stacking the articles 300 in the bag 100 is to arrange them with their side surfaces 310 parallel with the bottom 101. The package 1 can then comprise one stack of absorbent articles 300 or two stacks of absorbent articles 300 next to each other.

To manufacture a package 1 according to the disclosure, the at least one stack 201, 202 of absorbent articles 300 is arranged in a bag 100 as described above, wherein the bag has a closed bottom 101, an open top 102, and a peripheral wall 103 between the bottom 101 and the top 102. The inner surface top portions of the peripheral wall 103 provided with the adhesion layer 150 are thereafter adhered to one another as described above to obtain a closed top 102.

It will be appreciated that the above description is merely exemplary in nature and is not intended to limit the present disclosure, its application or uses. While specific examples have been described in the specification and illustrated in the drawings, it will be understood by those of ordinary skill in the art that various changes may be made and equivalents may be substituted for elements thereof without departing from the scope of the present disclosure as defined in the claims, the mixing and matching of features, elements and/or functions between various examples is expressly contemplated herein so that one of ordinary skill in the art would appreciate from this disclosure that features, elements and/or functions of one example may be incorporated into another example as appropriate, unless described otherwise, above. Moreover, many modifications may be made to adapt a particular situation or material to the teachings of the present disclosure without departing from the essential scope thereof. Therefore, it is intended that the present disclosure not be limited to the particular examples illustrated by the drawings and described in the, but that the scope of the present disclosure will include any embodiments falling within the foregoing description and the appended claims.

Reference signs mentioned in the claims should not be seen as limiting the extent of the matter protected by the claims, and their sole function is to make claims easier to understand.

1. A package comprising a bag and at least two absorbent articles arranged in the bag the bag being made of a first paper material,
wherein the bag has a sealed bottom, a top seal, and a peripheral wall extending between the bottom and the top seal, the peripheral wall comprising a front wall, an opposite rear wall, first and second side walls connecting the front and rear walls, a first top surface extending between the top seal and the front wall, and a second top surface extending between the top seal and the rear wall,
wherein the top seal has been formed by adhering inner surface top portions of the peripheral wall to one another, and
wherein the package further comprises at least one adhesive patch configured to hold the top seal folded onto one of the first and the second top surfaces.

2. The package according to claim 1, wherein the at least one adhesive patch comprises a non-adhesive front surface and a back surface, wherein an adhesive layer is provided on at least a portion of the back surface.

3. The package according to claim 1, wherein a first portion of the at least one adhesive patch is adhered to the top seal and wherein a second portion of the at least one adhesive patch is adhered to the one of the first and second top surfaces onto which the top seal is folded, and/or to one of the front and rear walls.

4. The package according to claim 1, wherein a first portion of the at least one adhesive patch is adhered to the top seal, and a second portion of the at least one adhesive patch is adhered to one of the first and second side walls.

5. The package according to claim 4, wherein a third portion of the at least one adhesive patch is adhered to the other one of the first and second side walls.

6. The package according to claim 3, wherein the at least one adhesive patch comprises a non-adhesive gripping portion configured to be gripped by a user for releasing at least the second portion of the at least one adhesive patch from the bag.

7. The package according to claim 1, wherein the at least one adhesive patch comprises a first adhesive patch and a second adhesive patch.

8. The package according to claim 7, wherein the first adhesive patch is attached to the top seal close to the first side wall, and wherein the second adhesive patch is attached to the top seal close to the second side wall.

9. The package according to claim 1, wherein the adhesive layer is a self-adhesive layer.

10. The package according to claim 9, wherein the self-adhesive layer is configured to allow resealing of the bag by the at least one adhesive patch.

11. The package according to claim 1, wherein the top seal together with the first and/or second top surfaces of the package form a planar or an essentially planar surface extending in parallel with the sealed bottom when the at least one adhesive patch holds the top seal folded onto one of the first and the second top surfaces.

12. The package according to claim 1, wherein the at least one adhesive patch comprises a backing material in the form of a second paper material.

13. The package according to claim 1, wherein the first paper material of the bag has a basis weight between 50 and 90 g/m2 as measured according to the ISO 536 standard.

14. The package according to claim 1, wherein the sealed bottom of the bag is formed by folding and adhering a portion of the paper material to form a flat and rectangular bottom.

15. The package according to claim 1, wherein the at least two absorbent articles are arranged as at least one stack of absorbent articles.

16. The package according to claim 15, wherein the at least one stack of absorbent articles comprises two stacks of absorbent articles.

17. The package according to claim 15, wherein the at least one stack of absorbent articles comprises a single stack of absorbent articles.

18. The package according to claim 1, wherein the bag is opaque and the absorbent articles, such as a wrapper of individually wrapped absorbent articles or the absorbent articles, cannot be seen through the bag.

19. The package according to claim 1, wherein the absorbent article is a disposable absorbent article such as a diaper, or an absorbent pad such as an incontinent shield or a sanitary napkin, or a washable and reusable absorbent article.
